(19) United States
(12) Patent Application Publication
(10) Pub. No.: US 2019/0290361 A1
(43) Pub. Date: Sep. 26, 2019

(54) IMPLANT DESIGN AND COMPUTER ASSISTED SURGERY

(71) Applicant: THINK SURGICAL, INC., Fremont, CA (US)

(72) Inventors: Stan G. SHALAYEV, Fremont, CA (US); In K. MUN, Fremont, CA (US); Micah FORSTEIN, Fremont, CA (US); Kyle KUZNIK, Fremont, CA (US); Timothy PACK, Fremont, CA (US)

(21) Appl. No.: 16/301,674

(22) PCT Filed: May 8, 2017
(86) PCT No.: PCT/US2017/031506

§ 371 Date: Nov. 14, 2018

Related U.S. Application Data
(60) Provisional application No. 62/336,945, filed on May 16, 2016.

(51) Int. Cl. A61B 34/10; A61B 17/86; A61B 34/30; A61F 2/30

(52) U.S. Cl. CPC A61B 34/10; A61B 17/86; A61B 34/30; A61F 2/34; A61F 2/30942; A61B 2034/102; A61F 2/30734

(57) ABSTRACT

A method of creating an augmented implant for a subject's bone or joint is provided. A virtual bone or joint model of the subject's bone or joint is obtained, the bone or joint model inclusive of bone property data for the subject's bone or joint including at least topology, density, and microarchitecture data. At least one of a size, a type, a geometry and a position for a virtual implant model is determined with respect to the bone or joint model to replace the region for removal using a processor. A stability region in the bone or joint surrounding the position of the implant model is located based on the bone or joint property data. The implant model is augmented with one or more stability features to interact with the stability region to improve implant stability using said processor or another processor. The augmented implant is so created.

### CROSS-REFERENCE TO RELATED APPLICATION

This application claims priority of U.S. Provisional Patent Application Ser. No. 62/336,945 filed May 16, 2016, which is incorporated herein by reference.

### TECHNICAL FIELD

The present invention generally relates to the field of computer assisted surgery, and in particular to the design of patient specific implants for use with computer assisted surgical systems.

### BACKGROUND

Patient specific implants (PSIs) are devices designed and manufactured to precisely conform to at least a portion of a patient's anatomy to replace an element of an anatomical region. The potential advantages of PSI include reduced overall costs, patient specific intervention, utilize preoperative planning, reduce surgery time, and improved surgical outcomes.

An important consideration for any surgical procedure is the stability and osseointegration of the PSI with the surrounding native bone. In particular, osseointegration is a key factor affecting implant longevity and stability, especially for cementless type implants. Micro-motion and the various loading conditions (i.e. axial loads, weight bearing, and shearing loads) between the implant and the bone interface may adversely affect osseointegration, which can potentially result in instability and revision surgery. Although various methods have been described for incorporating special design features to the PSIs to improve implant stability (e.g. keel design, keel location, peg designs, peg locations), patient specific bone properties such as growth factor levels, bone density, bone quality, and bone architecture vary significantly throughout different regions of the bone on a micro and sub-micro scale (i.e. 100 μm and 1 μm). In addition, the structure of the bone naturally changes after the surgical procedure due to bone remodeling based on external loading cues and remodeling time dependent biological in vivo processes. As current PSIs are specially designed to improve implant stability based on pre-surgical bone properties on a macro scale, the design may benefit by evaluating the varying bone properties surrounding the placement of the implant on a micro scale and below. Further, by simulating bone re-modelling during the design of an implant, the user may adjust or modify the implant to take advantage of the natural re-structuring of the bone that occurs in vivo to support optimal implant placement. While the use of autologous bone chips and cadaver bone surfaces can promote osseointegration, the processing of these materials is complex and always prone to contamination. Additionally, the dimensions and strength properties of implants formed of natural bone are inherently limited.

Equally important, the preparation of the bone to receive the implant requires extreme precision and accuracy to exploit the bone properties at such a small scale. Although patient specific jigs are often fabricated with the PSIs to prepare the bone, the conformance of the jigs with the patient's anatomy is not always accurate or structurally optimal. A surgeon may have to guess or estimate the precise positioning of the jig on the patient's anatomy due to unforeseen irregularities on the bone, osteophytes, image and processing errors, manufacturing errors, and discrepancies between pre-operative image measurements and intra-operative anatomical measurements. Patient specific jigs also lack the structure to aid in creating bone cuts having unique geometries (e.g. bone cuts requiring multiple shapes as a function of bone depth).

Thus, there is a need for a system and method to aid a user in designing an implant that is specific to a patient's varying bone structure and properties to improve implant stability and osseointegration. There is a further need to provide systems and methods to precisely fabricate the implant and prepare the patient's anatomy according to the design of the implant.

### SUMMARY OF THE INVENTION

A method of creating an augmented implant for a subject's bone or joint is provided for instance when the subject's bone has a required region for removal. A virtual bone or joint model of the subject's bone or joint is obtained, the bone or joint model inclusive of bone property data for the subject's bone or joint including at least topology, density, and microarchitecture data. At least one of a size, a type, a geometry and a position for a virtual implant model is determined with respect to the bone or joint model to replace the region for removal using a processor. A stability region in the bone or joint surrounding the position of the implant model is located based on the bone or joint property data. The implant model is augmented with one or more stability features to interact with the stability region to improve implant stability using said processor or another processor. The augmented implant is so created.

### BRIEF DESCRIPTION OF THE DRAWINGS

The present invention is further detailed with respect to the following drawings that are intended to show certain aspects of the present of invention, but should not be construed as limit on the practice of the invention, wherein:

FIG. 1 is a flowchart of a high-level method for designing an implant in accordance with embodiments of the invention;

FIG. 2 is a flowchart of a method for designing an implant by utilizing simulation models in accordance with embodiments of the invention;

FIG. 3A is a side view of a designed retaining component for anchoring an implant and FIG. 3B is a cross-section thereof in accordance with embodiments of the invention;

FIG. 4 illustrates a robotic surgical system to prepare the bone for receiving an implant in accordance with embodiments of the invention;

FIG. 5 illustrates the progression of designing an implant for a craniotomy procedure in accordance with embodiments of the invention;

FIGS. 6A and 6B illustrate the progression of designing an implant for acetabular reconstruction in accordance with embodiments of the invention;

FIGS. 7A-7C illustrate the final implant design for the acetabular reconstruction, where FIG. 7A is a back view of the implant, FIG. 7B is a detailed view of a first portion of the implant, and FIG. 7C is a cross-section view of a second portion of the implant in accordance with embodiments of the invention;

FIGS. 8A-8D illustrate an implant design for an ACL retaining total knee arthroplasty procedure, where FIG. 8A is an anterior elevation view, FIG. 8B is a perspective view thereof, FIG. 8C is a first cross-section view thereof and FIG. 8D is a second cross-section view thereof in accordance with embodiments of the invention;

FIGS. 9A-9F illustrates an example of designing an implant for a spine surgery application, where FIG. 9A is a sagittal view of a portion of a vertebrae model having a vertebral body with cancerous tumors, FIG. 9B depicts a density map superimposed on the spine model, FIG. 9C depicts a load/stress map of a vertebra, FIG. 9D depicts the microarchitecture of a cross-section of a vertebral body, FIG. 9E depicts a final implant design, and FIG. 9F depicts the final position of the final implant in the spine, all in accordance with embodiments of the invention.

### DETAILED DESCRIPTION OF THE INVENTION

The present invention has utility as a system and method for designing an implant that improves bone-to-implant stability and osseointegration. Intraoperatively, a computer-assisted surgical system may prepare the anatomy and aid in implant placement to exploit the stability and osseointegration design features of the implant. The following description of the various embodiments of the invention is not intended to limit the invention to these specific embodiments, but rather to enable any person skilled in the art to make and use this invention.

It is to be understood that in instances where a range of values are provided that the range is intended to encompass not only the end point values of the range but also intermediate values of the range as explicitly being included within the range and varying by the last significant figure of the range. By way of example, a recited range from 1 to 4 is intended to include 1-2, 1-3, 2-4, 3-4, and 1-4.

Reference is made herein to the replacement of anatomical regions illustratively including the hip joint, knee joint, spine and portions of the skull. As these are illustrative examples, it should be understood that the present invention may be applied to other bones and joints found within the body and may be implemented in other medical fields including neurosurgery, hand and foot surgery, maxillofacial surgery, plastic surgery, spine surgery, orthopedics, oncology, and dentistry applications. As used herein, a subject is defined as a human, a non-human primate; or an animal of a horse, a cow, a sheep, a goat, a cat, a rodent and a bird; or a cadaver of any of the aforementioned.

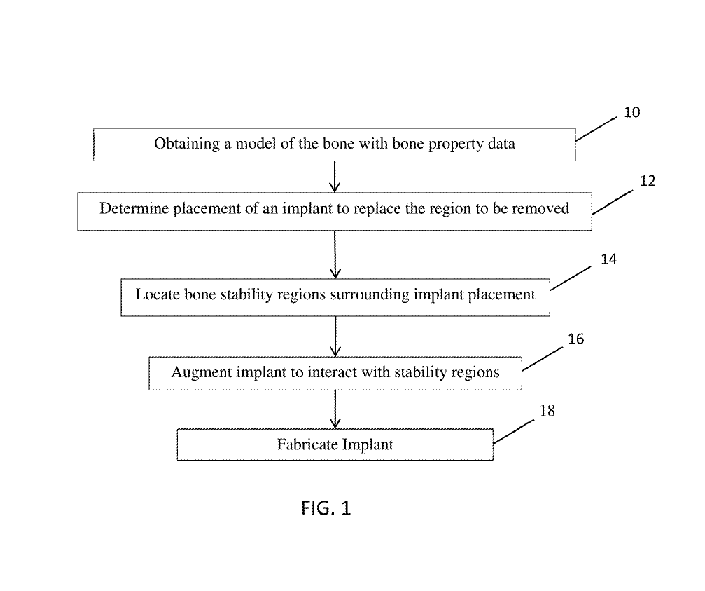

Embodiments of the present invention describe a system and method for designing an implant with features to improve the stability and osseointegration of the implant with the native bone. With reference to the figures, FIG. 1 is a flowchart of a high-level overview of a method for designing an implant in accordance with embodiments of the invention. A virtual bone model of the target bony anatomy or an element thereof is obtained that contains bone property data such as topology, bone density, and bone microarchitecture data (Block 10). At least one of a size, type, and geometry of a virtual implant model is determined and positioned on the bone model (Block 12). The implant is initially designed and positioned to replace a region of bone that requires removal or to restore the biomechanics of a subject's joint. Stability regions surrounding or adjacent to the implant model are located by evaluating the bone property data (Block 14). One or more features are augmented to the implant model to interact with the stability regions to improve the stability and to promote osseointegration of the implant with the bone due to an improved interface (Block 16). The final implant design is then fabricated for use in surgery (Block 18).

The bone model is obtained (Block 10) by generating a three-dimensional (3-D) bone model from an image data set of the subject's anatomy. The image data set may be collected with an imaging modality such as computed tomography (CT), dual-energy x-ray absorptiometry (DEXA), magnetic resonance imaging (MRI), X-ray scans, ultrasound, or a combination thereof. The 3-D bone model(s) are readily generated from the image data set using medical imaging software such as Mimics® (Materialise, Plymouth, Mich.) or other techniques known in the art such as the one described in U.S. Pat. No. 5,951,475.

Bone density data may be mapped to their corresponding locations on the bone model or built directly therein during the 3-D model generation using techniques known in the art. For example, relative bone density data in the form of CT values or Hounsfield units (HU) collected during a CT scan are retained during the generation of the bone model such that the CT values, or a corresponding metric (e.g. degree of brightness), is displayed with the bone topology. Hounsfield units for trabecular and cortical bone have been measured in the range of 100-350 HU for fine trabecular bone, 350-700 HU for the porous crestal layer of cortical bone and trabecular bone, 700-1200 HU for crestal cortical bone and coarse trabecular bone, and greater than 1200 HU for compact cortical bone. Although, the values are also dependent on patient specific factors such as age, weight, BMI, and bone pathology. Therefore, the relative bone density values may be converted or normalized into actual bone density values based on mathematical models, interpolating values from an imaged phantom having known density values, or using statistical models from a database of imaged specimens with known density values. However, it will become apparent that either relative bone density values (i.e. comparing the density of one bone region to another bone region from the same imaging scan) or actual bone density values can be used to improve the implant design.

Bone mineral density data in the form of T-scores or Z-scores may also be collected by with a 2-D or 3-D DEXA scan. A 3-D DEXA image data set can generate the bone model having the T-score or Z-score values bone density values incorporated directly therein. Other methods of incorporating bone density data includes the fusion of multiple image data sets to map the bone property data from one image data set to another (e.g. multi-view 2-D DEXA density values mapped to bone topology generated from a CT scan).

Microarchitecture data is obtained using a high-resolution imaging modality such as high-resolution 3D peripheral quantitative micro-CT (HR-pQCT), high-resolution magnetic resonance imaging (HR-MRI), or other imaging modalities capable of imaging the bone on a micrometer scale or below. An imaging system and method for obtaining images at this scale on human subjects is described in U.S. Pat. No. 7,840,249. The microarchitecture data includes the trabecular structure of the bone which provides another indicator of bone health, quality and structural integrity. A 3-D model of the microarchitecture can be generated from the high-resolution image data sets. If the bone topology was generated with a different imaging modality, the microarchitecture data is mapped to the bone topology using known anatomical landmarks between the two data sets. In some instances, the high-resolution imaging modality may be restricted to a particular field of view. In which case, the user can determine what field of view is most applicable, or multiple scans at varying locations is performed, pieced together known as “overlay grid”, reconstructed and mapped to the topology data.

The user is able to view and manipulate the bone model and bone property data in a pre-operative planning software program having a graphical user interface (GUI). The GUI includes widgets and other tools for manipulating the bone model and designing an implant. With the bone model, the user first determines at least one of a size, type, geometry and position for an initial implant (Block 12). For clarity, the initial implant refers to an implant design that replaces a region of bone requiring removal or to restore the biomechanics of a subject's joint. In other words, the initial implant design serves the purpose for the surgical procedure. In a particular embodiment, the user designs the initial implant using a library of generic implant models labelled by manufacturer type and size. The generic implant models may be in the form of computer aided design (CAD) models or equivalent. The user can select an implant model from the library and optimally position the implant model relative to the bone model such that the implant model replaces the region of bone to be removed. The user may re-select different manufacturer types and sizes before a desired position is determined to achieve the primary goal of the surgical procedure. The generic implant models may have modifiable surface where the user can extrude or cut-out portions of the generic implant to generate the initial implant design. In another embodiment, the GUI includes tools to design the initial implant without a generic implant model. Splines, lines and generic shapes with or without modifiable meshed surfaces are used to define the geometry of the implant model to replace the region to be removed. A library of modifiable shapes resembling typical structures of a particular implant (e.g. a peg or a keel of a tibial base plate implant) may also be provided in the GUI. In yet another embodiment, geometric data about the bone model may be extracted to aid in the initial implant design. The topology of the bone model may be extracted to define the natural contours and curvature of the native bone. The contours and curvatures may define at least a portion of the implant shape to match the actual bone topology. For example, when designing a knee implant, the contour of the medial and lateral condyles of the distal femur may be extracted to aid in defining the geometry of the outer surface of the femoral knee implant. In a specific embodiment, the library of generic implant models, the tools for defining a desired shape (i.e. splines, lines and shapes), and the geometry extraction tools are all available to the user in the GUI. These widgets and tools can also be used for augmenting the implant with the stability and osseointegration features.

After the initial implant is designed and the position is determined on the bone model, the user locates bone stability regions surrounding the implant design (Block 14). Bone stability regions are generally defined as regions having relatively higher bone quality. Bone quality is a function of bone density, bone microarchitecture, and in specific embodiments, loading conditions experienced on the surrounding bone after implant placement that may prompt bone remodeling and affect bone density. A bone density metric such as a degree of brightness, numbers, or shading may indicate potential stability regions at a macroscopic scale (i.e. a majority of the bone model in view). To closely inspect the bone quality, the GUI includes a zoom tool to reveal the bone microarchitecture. Trabecular bone is generally organized according to Wolff's Law in which the individual trabeculae are oriented perpendicular to the regions experiencing loads. The user may manually assess the microarchitecture in terms of different parameters including trabecular bone volume density, mean trabecular area, mean trabecular perimeter, individual diameters of trabeculae, and the density of individual trabeculae in terms of relative microCT values. These parameters may also be assessed semi-automatically or automatically with a software module programmed with the GUI to display the values directly to the user. For example, if the user highlights a specific region, the GUI displays the trabecular bone density. If the user clicks on a particular trabeculae or a set of trabeculae, the GUI may display the diameter, length, and density of the trabeculae or an average thereof for a set of trabeculae.

Once suitable anatomical element candidates for the optimal stability regions are located, the user augments the initial implant design with one or more stability features to interact with the stability regions (Block 16). The stability features may vary in length, diameter, width, bone depth, and angle as desired by the user. For example, a stability feature may have a cylindrically shaped base with an eyelet at its end where the entire stability feature is inserted into an inner portion of the bone such that the bone completely surrounds the stability feature. The stability features may respect 3-D anatomical relationships with the subject's anatomy by designing interdigitated areas with the implant and the bone. In addition, osseointegration features are added to the initial implant design and stability features to interact with the native bone to promote osseointegration. The osseointegration features are multidimensional and/or directional in the general form of ridges, grooves, channels, and projections on the micrometer and sub-micrometer scale. The osseointegration features are designed to stimulate bone formation, osteoclast activities, osteoblast differentiation, or promote osteoblast migration. In particular, the osseointegration features promote cell migration from regions of high bone quality to regions of low bone quality, especially in locations where the initial implant design causes a redistribution of loads on the bone as described below. The user may further design the osseointegration features to promote cell migration in certain areas and bone formation in others. By adjusting the width, depth and spacing of the grooves, ridges, channels, or projections along the implant or the stability features, the migration, differentiation, and bone deposition of the cells may be controlled. The osseointegration features may further include channels prefabricated with additive or subtractive technologies that exist within the bulk of the implant or stability features to promote directional and multidimensional osseointegration into or out of the channels. For instance, a channel bored through the implant may receive a section of bone therewithin such that the channel surrounds the section of bone.

Bone conduction, the growth of bone on an implant surface (synonymously referred to herein and in the literature as osseoconduction) depends on the action of differentiated bone cells. These cells may originate either in pre-existing preosteoblasts/osteoblasts that are activated by surgical trauma or in cells recruited from primitive mesenchymal cells by osteoinduction. Various types of bone growth factors are necessary for bone formation. Furthermore, bone growth, including bone conduction, does not occur without a proper blood supply and therefore growth factors that are both angiogenic and mitogenic that can be added to the implant or stimulations at the implant-body interface appear to promote osseointegration in the present invention. Growth factors that regulate bone tissue in one way or another include insulin-like growth factor (IGF I, II), vascular endothelial growth factor (VEGF), fibroblast growth factor (FGF), TGF-β, platelet-derived growth factor (PDGF) and combinations thereof are illustrative of growth factors that physically coat or are covalently bonded to the surfaces of the implant to which osseointegration is desired. It is appreciated that a slow release matrix contains a growth factor is operative herein; such matrices include Choukroun's platelet-rich fibrin (PRF).

Bone conduction, the growth of bone on the surface of the implant is disfavored on certain materials such as copper and silver, yet occurs on material common to orthopedic surgery such as stainless steel, titanium, titanium alloys, hydroxyapatite, and combinations thereof. As a result an implant can be stabilized by coating portions of an implant with any of the aforementioned. It is appreciated that even in instances when osseointegration does not occur to and appreciable degree, bone conduction by forming a new layer bone at the interface with an implant surface imparts a desired implant stabilization according to the present invention. The bone-implant interface in osseointegration according to the present invention typically has an amorphous layer from 20 to 500 nm thick. Collagen and calcified tissue are typically found in this interface.

In a particular embodiment, an implant augmentation is formed from with a surface material containing a minority phase of a soluble metal such as silver, magnesium, or zinc. Titanium with up to 4.5 atomic percent silver is exemplary thereof. Upon subjecting the surface material to mineral acid, the soluble metal is preferentially dissolved leaving a controlled level of porosity. Through control of porosity in the implant augmentation surface, osseointegration is promoted. Additionally, the percentage of soluble material can vary at different locations of the implant, stability or osseointegration feature to create porosities that correspond to a given microarchitecture.

In a particular embodiment, during implant augmentation, the GUI may constrain the initial implant geometry, position and orientation such that the implants primary surgical purpose is not compromised. After the final implant is designed and completed, the user sends the design or fabrication instructions to a manufacturer to fabricate the final implant (Block 18). For clarity, the final implant refers to the initial implant design including the stability and osseointegration features.

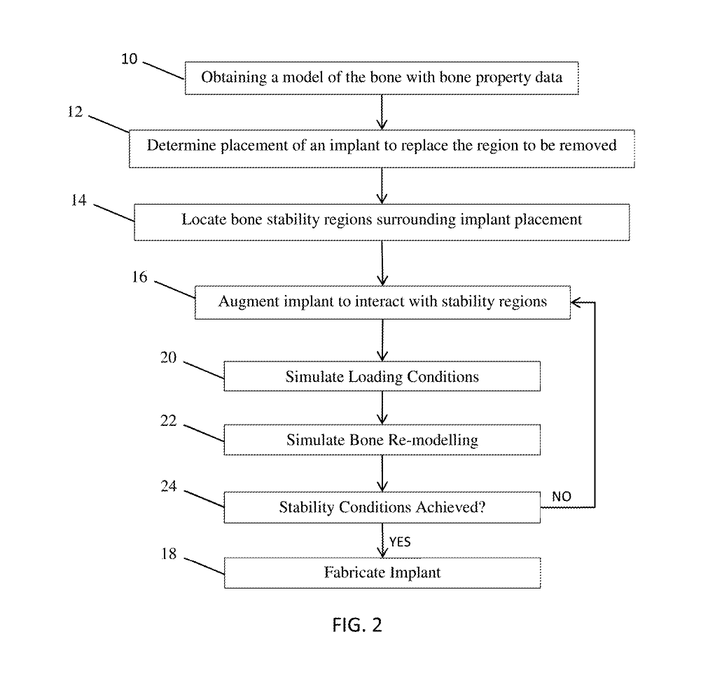

In a specific embodiment, with reference to FIG. 2, loading conditions are simulated after the user augments the initial implant design with the one or more features (Block 20). Finite element analysis (FEA) is executed that models and simulates the normal or worst case physiological loads that the implant will experience in vivo. The FEA may simulate loading conditions for different forms of physical activity such as standing, walking, running, carrying loads, pushing and pulling loads, chewing, and the like using kinematic modelling. An illustrative method of performing FEA on a bone and an implant is described in M. A. Kumbhalkar, “Modeling and Finite Element Analysis of Knee Prosthesis with and without Implant”, Universal Journal of Computational Mathematics 1(2): 56-66, 2013. A method of performing subject specific FEA with incorporated density values acquired from a CT scan is described in R. Blanchard, “Intravoxel bone micromechanics for micoCT-based finite element simulations”, Journal of Biomechanics 46 (2013) 2710-2721. In a particular embodiment, the FEA simulates the physiological loads experienced on individual trabeculae or sets of trabeculae in the bone microarchitecture. A distribution map of the loading conditions is readily displayed to indicate the bone regions that will experience loads after implant placement. The GUI may have options to view the loading conditions for different forms of physical activity or display an average of the compiled loads experienced during a wide variety or spectrum of the physical activities.

The user may modify the stability features and perform additional FEA until the features account for the new load distributions. The stability feature modifications may be performed automatically by the planning software where the FEA simulations and modifications are run iteratively. If the user is targeting a particular stability region due to its enhanced bone quality, the user may define constraints in the FEA, where the simulations may modify the size and geometry of the feature while maintaining at least a portion of the feature within the particular stability region.

In a specific embodiment, the FEA is conducted on the initial implant design prior to the augmentation. Bone re-modelling simulations are conducted on the cortical bone and microarchitecture (Block 22) to determine how the initial implant design affects bone re-modelling on the surrounding native bone. A method for simulating bone re-modelling in trabecular bone according to different loading patterns is described in S. M. Banijamali, “Effects of Different Loading Patterns on the Trabecular Bone Morphology of the Proximal Femur using Adaptive Bone Remodeling”. By adapting this method to simulate the loading patterns generated on the surrounding bone from the new implant design, a new microarchitecture of the bone is provided to locate new stability regions. The simulation of the FEA and bone re-modelling may also be conducted after the user has augmented the implant with stability and osseointegration features as outlined in FIG. 2 to determine how those features affect the loading conditions and microarchitecture of the bone.

In a particular embodiment, the bone quality is assessed before and after the simulations to locate regions of high bone quality pre-implantation and the regions of high loading and bone re-modelling post-implantation. The areas of high bone quality pre-implantation having reduced loads post-implantation are potential candidates for promoting cell migration to regions that may experience higher loads post-implantation. The stability and osseointegration features may therefore be designed to interact and promote cell migration from the pre-implantation high quality regions to a region that will experience higher loads and bone re-modelling post-implantation. In the short-term, the high bone quality regions can directly interact with the stability and osseointegration features to firmly stabilize the implant. In some inventive embodiments, autologous bone chips are packed into recesses in the implant to seed and otherwise foster osseointegration. Overtime, as the bone experiences the shifted loads at a new region, cells can migrate from the high-quality region along the surface or through the osseointegration features to the new loading regions. For cementless implants, this is highly desirable for the short-term and long-term success of the implant. Since the high-quality region prior to implant placement will experience less loads after implant placement, Wolff's law predicts this area to naturally re-model with less trabecular bone. It is notable, that the user must determine whether this transfer of loads is causing stress-shielding in key locations along the length or surrounding implant that may be detrimental to the overall bone-to-implant stability. Stress-shielding is a common phenomenon that occurs when loads are transferred and/or redistributed to other portions of the bone and in certain cases, depending on the location of the stress-shielding, can have a negative impact on bone-to-implant stability.

In another embodiment, the user may identify stability regions having high bone quality pre-implantation that experience less loading post-implantation. The initial stability of the implant may be improved by designing stability features to interact with this region because the probability of micro-motion is reduced. Due to the initial high bone quality and reduced stresses after implantation, the probability of osseointegration and bone formation is favorable. The bulk porosity of the implant features in these regions can be increased to promote ingrowth and osseointegration since a more durable structure to withstand higher loads is not required.

The FEA and bone remodeling simulations can be run iteratively until the stability conditions are achieved (Block 24). In one embodiment, the stability conditions may be achieved based on the user's discretion after evaluating the results of the simulations. In another embodiment, the stability conditions may be achieved when a desired proportion of the stability features interact with the stability regions located post-simulation. For example, pre-simulation the user may augment a stability feature in which 95% of the feature interacts with an identified stability region. Post-simulation, the bone may have re-modelled or the loads may have redistributed in which only 50% of the stability feature now interacts with the pre-simulated stability region. The user, or the planning software, may modify the stability feature until greater than 80% of the stability feature interacts with a post-simulated stability region, at which time the bone stability conditions are achieved. The proportion of the feature interacting with the stability region may be set by the user (e.g. 50%-95%). In another embodiment, the stability conditions are achieved based on an expected or simulated amount of micro-motion. For example, loading and bone-remodeling simulations are performed after the implant is augmented with stability and osseointegration features. The planning software may highlight regions experiencing stresses above a specified micro-motion threshold. Subsequently the user, or planning software, may modify the features until the stresses drop below the micro-motion threshold. The micro-motion threshold may be set by the user. In another embodiment, a time-dependent bone remodeling simulation may determine the time required for the bone to remodel and in what locations this is likely to occur wherein the stability conditions are achieved when the bone has sufficiently remodeled around the osseointegration features. Once the stability conditions are achieved the final implant design or fabrication instructions are sent to a manufacturer for fabrication (Block 18). It should be appreciated that a combination of the aforementioned subjective and objective criteria may be used to determine when the stability conditions are achieved.

### Retaining Components

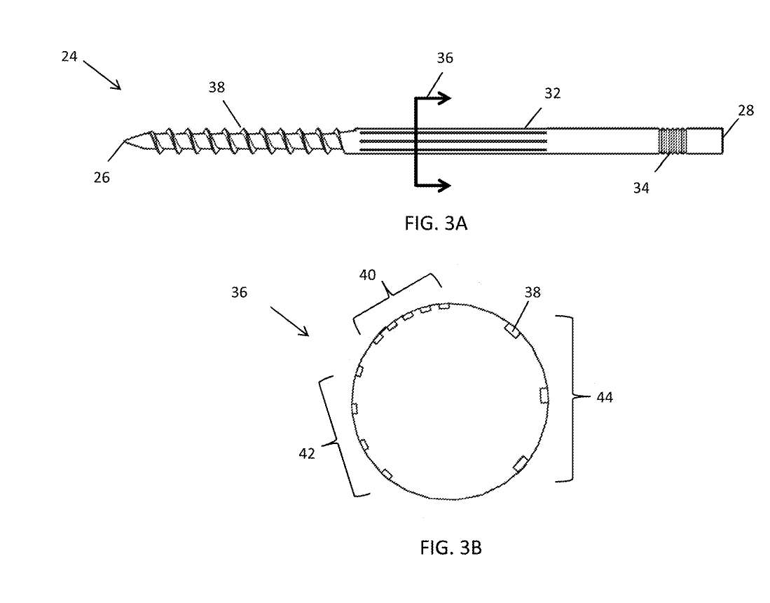

In a specific embodiment, the user may design one or more retaining components that interact and anchor the implant. The retaining components are designed to fix the implant into position on the bone and may include for example bone pins, bone screws, and bone nails. The retaining component may also include stability and osseointegration features designed by the user according to the bone property data, the FEA simulations, and/or the bone remodeling simulations. With reference to FIGS. 3A and 3B, an example of a retaining component 24 is shown. FIG. 3A is a side view of the retaining component 24. The retaining component 24 may resemble a bone screw having a distal end 26, a proximal end 28, a threaded portion 30, and osseointegration features 32 and 34. The distal end 26 is configured to engage the bone such as a pointed tip, or a cutter having teeth like an end-mill. The proximal end 28 may be a shank or have a blunt end to aid in driving the retaining component 24 into the bone. The threaded portion 38 may include flutes for grasping the bone to aid in inserting the component 24. The threaded portion 38 may have a smaller diameter than the remaining portion such that osseointegration features 32 and 34 have direct contact with the bone when inserted.

FIG. 3B (osseointegration features not to scale) depicts a cross-section view 36 of the component 24 to show examples of osseointegration features. The osseointegration features 32 include channels 38 of varying depth and distribution extending along the length of the component 24. For example, a first osseointegration feature section 40 illustrates the channels spaced around 1-5 micrometer apart with a depth of less than 1 micrometer. A second osseointegration feature section 42 illustrates channels spaced around 5-10 micrometers apart with a depth of 1-5 micrometers. A third osseointegration feature section 44 illustrates channels space around 10-20 micrometers apart with a depth of 5-10 micrometers. In another embodiment, the channels are replaced with projections extending along the length of the component 24 and projecting away from the surface of the component 24. If the osseointegration features are projections, the component 24 is inserted into the bone without a rotational action such that the projections crush directly into the bone to ensure maximal bone-to-implant contact area. In some embodiments, the projections are seeded with growth factor, autologous bone chips, or a combination thereof. In that case, the retaining component may resemble a bone nail having no threaded portion. Another set of osseointegration features 34 are shown oriented around the radial axis of the retaining component 24, which is useful if the bone microarchitecture remodels according to a radial load imposed on this portion of the retaining component 24. It should be appreciated that the overall dimensions of the retaining component is a function of the surgical application, amount of bone available, and the implant design.

In a specific embodiment, the retaining component has osseointegration features to promote cell migration and bone formation towards the implant. If the user identifies a high bone quality region that cannot be reached by an implant stability feature, the retaining component may drive through the high quality bone region and intercept with the implant. The osseointegration features on the retaining component may guide or provide a route and/or surgical placement trajectory to the implant to aid in bone formation and stability at the bone-to-implant interface.

According to the present invention osseointegration of an inventive implant occurs more rapidly by treating the implant with one or more to the following: a hydroxyapatite coating, roughened implants exposed surfaces on a scale that promotes osteoblast infiltration, growth of thicker than native oxides on exposed implant surfaces, and surface coating with growth factors. It is appreciated that orthopedic arthroplasty procedures that promote osseointegration include minimizing interfacial heat due to curing bone cement, tool contact with subject tissue with concurrent usage of a cooling agent, and slow force loading of the implant. According to the present invention interfacial implant movement is limited in some instances to less than 150 μm to limit soft tissue formation instead of osseointegration. Screw-type implants inserted using a minimally traumatizing techniques are well suited to promote osseointegration.

### Fabrication

It is contemplated that various manufacturing methods known in the art can fabricate the implant and retaining components including additive and subtractive manufacturing methods. Additive manufacturing methods include for example stereolithography, polyjet, fused deposition modeling, selective laser melting, selective laser sintering, and electron beam melting. The type of materials to fabricate the final implant generally depends on the surgical procedure. For instance, metal alloys to fabricate femoral knee and hip implants, ceramics for dental implants, and durable plastics for tibial liners and acetabular cup liners. In a specific embodiment, polyetherketoneketone (PEKK) is a desirable bone interfacing material due to its superior bone like properties and interaction with the native bone on a cellular level as described in T. Ganey, “Cell proliferation and vitality determination of osteoblasts on different materials and surface characteristics; interpretation of laboratory data”, Confidential OPM Report, March 2011. PEKK is an ideal material for implant regions that directly contact the bone to promote osseointegration and bone formation. In certain instances it may be desirable to fabricate the implant with two or more materials to achieve the desired performance criteria and improved stability. For example, a femoral knee implant may include an outer surface made of a metal alloy to provide a durable and smooth surface while the inner surface and the stability features are made of PEKK to interact with the bone. The interface between the two or more materials are adhered, interlocked, or otherwise integrated to maintain the integrity of the implant.

A desired roughness of the material and the fabrication of the osseointegration features on the micrometer and sub-micrometer scale may be created with highly precise additive or subtractive manufacturing or other manufacturing methods such as microfabrication etching, laser cutting, and stamping. In a particular embodiment, a stamp imprints the design of the feature on the implant. A negative match of the implant stamp may be fabricated for stamping the bone to create a negative pattern of the osseointegration feature to improve the bone-to-implant surface contact area. It should be appreciated that other surface characteristics of the implant can be altered to promote osseointegration including varying the surface and bulk porosity, and coating the surface with agents such as hydroxyapatite. In a particular embodiment, the concentration of surface coating agents may vary along the final implant to promote cell migration in certain areas and bone formation in other areas. The concentration, presence or absence of biological growth factors, differentiating factors, cell-adhesion factors, anti-cell adhesion factors and autologous bone chips may also vary in different locations on the final implant to control cell motility, fate and expression.

In a particular embodiment, the instructions for fabricating the implant are used to develop the instructions for a robotic surgical system to create the bone cuts. Since the geometry and position of the implant are designed with respect to the bone, the instructions used to create implant geometry correspond directly with the geometry that needs to be removed on the bone. The fabrication instructions provide the relative set of points that can be executed by the robotic surgical system intra-operatively. In another embodiment, the fabrication instructions provide a starting point for creating the robotic instructions, which can be modified by the user to define the orientations of a manipulator arm of the robotic system to gain access to particular points defined in the fabrication instructions. Additionally, the intersection between the bone model and the final implant design can designate which points in the cut-file are necessary and which points can be removed. For example, if a portion of the points for fabricating the implant do not intersect with the bone model, those points can be removed from the cut-file to improve the robotic cutting time. A robotic system for executing a cut-file is further described below.

### Bone Preparation and Implant Installation

To take full advantage of the finalized implant design and retaining component design, especially due to their micrometer and sub-micrometer attributes, the bone must be prepared to precisely receive the implant. In certain instances, the stability and osseointegration features need to align with the microarchitecture and stability regions located in the plan. Therefore, a computer-assisted surgical system capable of executing such precision is desirable. Examples of a computer-assisted surgical system include a 1-6 degree of freedom hand-held surgical system, an autonomous serial-chain manipulator system, a haptic serial-chain manipulator system, a parallel robotic system, or a master-slave robotic system, as described in U.S. Pat. Nos. 5,086,401, 7,206,626, 8,876,830 and 8,961,536, U.S. Pat. App. No. 2013/0060278, and PCT Intl. App. No. US2015/051713.

With reference to FIG. 4, a particular embodiment of a robotic surgical system 50 to prepare and/or install the implant is shown in the context of an operating room (OR). The surgical system 50 generally includes a surgical robot 52, a computing system 54, and a tracking system 56.

The surgical robot 52 includes a movable base 58, a manipulator arm 60 mounted to the base 58, an end-effector flange 62 located at a distal end of the manipulator arm 60, an end-effector assembly 64 removably attached to the flange 62, and a tool 66 removably assembled to the end-effector assembly 64. The base 58 may include a set of wheels 68 to maneuver the base 58, which may be fixed into position using a braking mechanism such as a hydraulic brake. The manipulator arm 60 includes various joints and links to manipulate the tool 66 in various degrees of freedom. The joints may be prismatic, revolute, or a combination thereof. The tool 66 may be any device to contact, perform work or install an implant on the subject's anatomy including for example a burr, a saw, an end-mill, a cutter, a laser engraver, forceps, a claw, electrocautery device, a drill, a pin driver, a reamer, an ultrasonic horn, or a probe. The tool 66 and manipulator are controlled by commands from the computing system 54 and tracking system 56.

The computing system 54 generally includes a planning computer 70 including a processor; a device computer 72 including a processor; a tracking computer 74 including a processor; and peripheral devices. Processors operate in system 54 to perform computations associated with the inventive method. It is appreciated that processor functions are shared between computers, a remote server, a cloud computing facility, or combinations thereof. The planning computer 70, device computer 72, and tracking computer 74 may be separate entities as shown, or it is contemplated that their operations may be executed on just one or two computers depending on the configuration of the surgical system 50. For example, the tracking computer 74 may have the operational data to control the manipulator 60 and tool 66 of the surgical system 50 without the need for a device computer 72. Or, the device computer 72 may include operational data to plan the surgical procedure and design the implant without the need for the planning computer 70. In any case, the peripheral devices allow a user to interface with the surgical system components and may include: one or more user-interfaces, such as a display or monitor 76; and user-input mechanisms, such as a keyboard 78, mouse 80, pendent 82, joystick 84, foot pedal 86, or the monitor 76 may have touchscreen capabilities.

The planning computer 70 contains hardware (e.g., processors, controllers, and memory), software, data and utilities that are dedicated to the implant design and planning of a surgical procedure, either pre-operatively or intra-operatively. This may include reading medical imaging data, segmenting imaging data, constructing three-dimensional (3D) virtual models, storing computer-aided design (CAD) files, providing the GUI and tools for implant and retaining component design, and generating surgical plan data and implant manufacturing instructions. The final surgical plan includes manufacturing instructions to fabricate the final implant design pre-operation, and intra-operative operational data for modifying a volume of tissue to receive the implant in the position and orientation defined in the plan, such as a set of points in a cut-file to autonomously modify the volume of bone, a set of virtual boundaries defined to haptically constrain a tool within the defined boundaries to modify the bone, a set of planes or drill holes to drill the retaining components in the bone, or a graphically navigated set of instructions for modifying the tissue. The data generated from the planning computer 70 is readily transferred to the device computer 72 and/or tracking computer 74 through a wired or wirelessly connection in the operating room (OR); or transferred via a non-transient data storage medium (e.g., a compact disc (CD), a portable universal serial bus (USB) drive) if the planning computer 514 is located outside the OR.

The device computer 72 may be housed in the moveable base 58 and contain hardware, software, data and utilities that are primarily dedicated to the operation of the surgical device 72. This may include surgical device control, robotic manipulator control, the processing of kinematic and inverse kinematic data, the execution of registration algorithms, the execution of calibration routines, the execution of surgical plan data, coordinate transformation processing, providing workflow instructions to a user, and utilizing position and orientation (POSE) data from the tracking system 56.

The tracking system 56 of the surgical system 50 includes two or more optical receivers 86 to detect the position of fiducial markers (e.g., retroreflective spheres, active light emitting diodes (LEDs)) uniquely arranged on rigid bodies. The fiducial markers arranged on a rigid body are collectively referred to as a fiducial marker array 88, where each fiducial marker array 88 has a unique arrangement of fiducial markers, or a unique transmitting wavelength/frequency if the markers are active LEDs. An example of an optical tracking system is described in U.S. Pat. No. 6,061,644. The tracking system 56 may be built into a surgical light 90, located on a boom, a stand, or built into the walls or ceilings of the OR. The tracking system computer 74 may include tracking hardware, software, data and utilities to determine the POSE of objects (e.g., bones B, surgical robot 52) in a local or global coordinate frame. The POSE of the objects is collectively referred to herein as POSE data, where this POSE data is readily communicated to the device computer 72 through a wired or wireless connection. Alternatively, the device computer 76 may determine the POSE data using the position of the fiducial markers detected from the optical receivers 86 directly.

The POSE data is determined using the position data detected from the optical receivers 86 and operations/processes such as image processing, image filtering, triangulation algorithms, geometric relationship processing, registration algorithms, calibration algorithms, and coordinate transformation processing. For example, the POSE of a digitizer probe 92 with an attached probe fiducial marker array 88d may be calibrated such that the probe tip is continuously known as described in U.S. Pat. No. 7,043,961. The POSE of the tool tip or tool axis of the tool 66 may be known with respect to a device fiducial marker array 88c using a calibration method as described in U.S. Prov. Pat. App. 62/128,857. The device fiducial marker 88c is depicted on the manipulator arm 60 but may also be positioned on the base 58 or the end-effector assembly 64. Registration algorithms are readily executed to determine the POSE and coordinate transforms between a bone B, a fiducial marker array 88, and a surgical plan, using the registration methods described in U.S. Pat. Nos. 6,033,415, and 8,287,522.

The POSE data is used by the computing system 54 during the procedure to update the coordinate transforms between the bone B, the surgical robot 52, and the surgical plan to ensure the surgical robot 52 accurately executes the surgical plan on the bone B. It should be appreciated that in certain embodiments, other tracking systems may be incorporated with the surgical system 50 such as an electromagnetic field tracking system or a mechanical tracking system. An example of a mechanical tracking system is described in U.S. Pat. No. 6,322,567. In a particular embodiment, the surgical system 50 does not include a tracking system 56 and a tracked digitizer probe 92, but instead employs a mechanical digitizer arm incorporated with the surgical robot 52 as described in U.S. Pat. No. 6,033,415, and a bone fixation and monitoring system that fixes the bone directly to the surgical robot 52 and monitors bone movement as described in U.S. Pat. No. 5,086,401 both of which are incorporated by reference herein in their entirety.

In a particular embodiment, the surgical system 50 prepares the bone to precisely receive the finalized implant and any retaining components. The cuts created on the bone provide a guide to precisely position and orient the implant onto the bone due to the unique position and orientation of the stability features. For instance, the final design is likely to have an asymmetrical shape with features projecting in unique directions. Since the bone cuts are a congruent match of those projecting features, the user does not have to guess or estimate the proper positioning of the implant on the bone, which is otherwise a problem with other subject specific implants due to unexpected bone geometries, osteophytes, and pre-operative anatomical measurement error. The variable cuts and any interdigitating features also respect the 3-D anatomical relationship between the implant and subject's anatomy.

In a specific embodiment, the surgical system 50 aids in the installment of the implant or the retaining components. Since the stability and osseointegration features are designed to target specific bone quality regions, manually installing the implant likely results in the misalignment of the micrometer and sub-micrometer features with the bone structure. To ensure the implant is precisely aligned according to the plan, the tool 66 is configured to grasp and install the implant or retaining components after the bone is prepared. The digitizer probe 92 may digitize the implant/retaining component to register their POSE with respect to the surgical system 50 and boney anatomy. The implant may include one or more registration features such as three or more divots to facilitate the registration of the implant to the surgical system 50. With the implant registered to the surgical system 50, the POSE and depth of the implant/retaining component and the features thereon are precisely positioned according to the plan. In a particular embodiment, the POSE of the osseointegration features is known with respect to the surgical system 50 and bony anatomy. The surgical system 50 may determine how many rotations of the retaining component is required to drill the component into the bone such that any particular osseointegration features thereon align with the trabecular bone structures identified during the implant design and surgical planning.

In a specific embodiment, the surgical system 50 aids in preparing negatively matching patterns on the bone corresponding to the osseointegration features of the implant. As with the stamp described above, the tool creates patterns on the bone to improve the bone-to-implant surface contact area. For instance, if the osseointegration features include a ridge-then-groove arrangement, then the tool may create a groove-then-ridge pattern (i.e. a negative match of the features) on the bone to form an interdigitating bone-to-implant interface. This may be accomplished with a finishing surface cut using the micro-structure of a tool 66. The tool 66 is fabricated with ridges and grooves corresponding to a negative match of the osseointegration features. The tool 66 is then passed along the surface of the bone to create the pattern. Multiple tools 66 having different structures for different parts of the implant can be fabricated at the same time as the implant fabrication. For more complex shapes, the surgical system 50 can control a temperature regulated laser etching tool to create the negative matching patterns on the bone.

### EXAMPLES

### Example 1: Implant Design for a Craniotomy Procedure

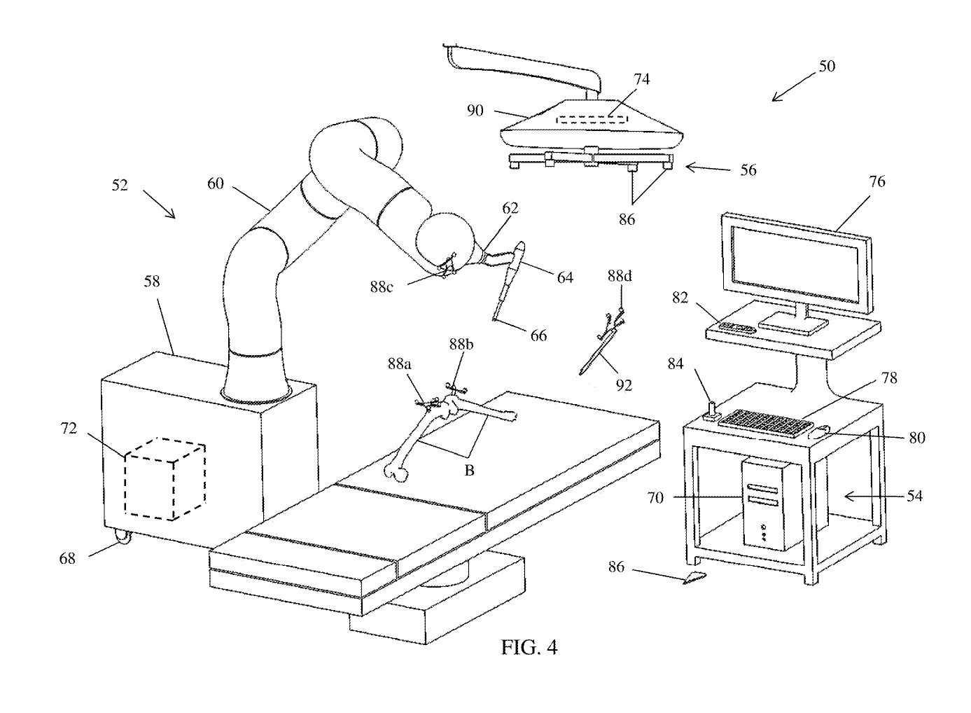

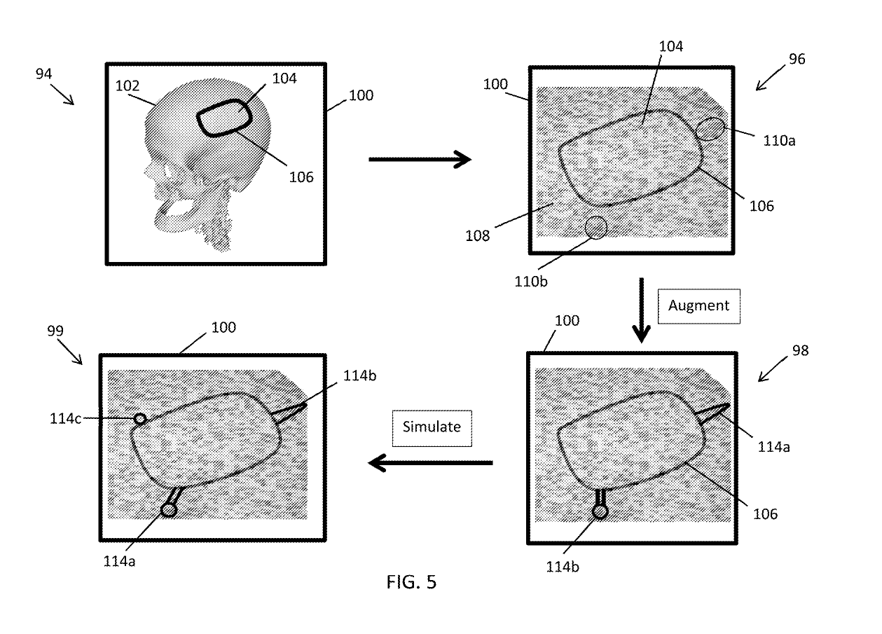

FIG. 5 illustrates the progression of designing an implant for a craniotomy procedure in accordance with the embodiments described herein. A model of the skull 102 is obtained and displayed in a GUI 100 as shown at 94. The skull model 102 may have been generated from a CT scan, while the inner brain tissue may have been generated from an MRI scan, with both scans being fused to plan the procedure in the GUI 100. The user determines a region of bone required for removal 104 to reach a targeted site in the brain. The geometry and amount of bone to be removed 104 is at the discretion of the surgeon, but it should be the least amount of bone that can be removed while maintaining the ability to reach the target site and conduct the procedure safely. The GUI 100 includes tools to design the initial implant 106 to replace the required region for removal 104. In an embodiment, the initial implant is designed with a point and click tool that allows the user to insert singular points at the desired outer boundary of the initial implant. Subsequently, the planning software automatically generates the implant volume and geometry by connecting the singular points with splines that follow the curvature of the bone using mathematical models such as non-uniform rational Bezier spline (NURBS). This ensures the curvature of the implant matches the natural curvature of the skull.

The initial implant area is zoomed-in so the user can identify stability regions surrounding the implant as shown at 96. A bone density map 108 of the skull 102 is displayed to the user in the form of brightness values, which provides the relative bone densities of the bone. The user highlights or labels the desired stability regions as represented by 110a and 110b and one or more stability features are augmented to the initial implant to interact with these stability regions 110.

Stability features 114 augmented to the initial implant are shown at 98. The user may design one stability feature 114 to have a parabolic form to interact with a greater portion of the higher bone quality. A second stability feature 114b may have a thin rectangular base to traverse less dense regions and a circular end to receive a retaining component that directly drives into the high bone quality region. The stability features 114 may be designed to overlay on top of the native bone, or the bone may be prepared with a partial inset to receive at least a portion of the stability features 114 within the inset. After the augmentation step, a simulation is run to determine if the final implant is properly stabilized based on the geometry of the implant, the geometry of the surrounding bone, and the loading conditions. The simulation may automatically update the position, size and shape of the augmentation features 114, as well as propose additional augmentation features 114c to support and stabilize the final implant as shown at 99. After the user approves the final implant design, the implant is fabricated for use in the craniotomy procedure.

### Example 2: Implant Design for Acetabular Cup Reconstruction

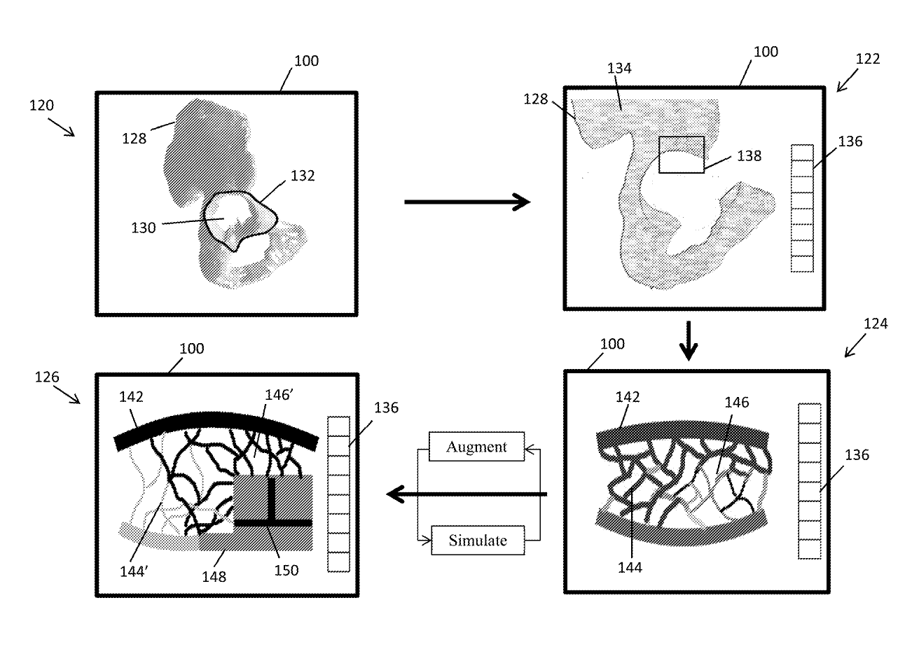

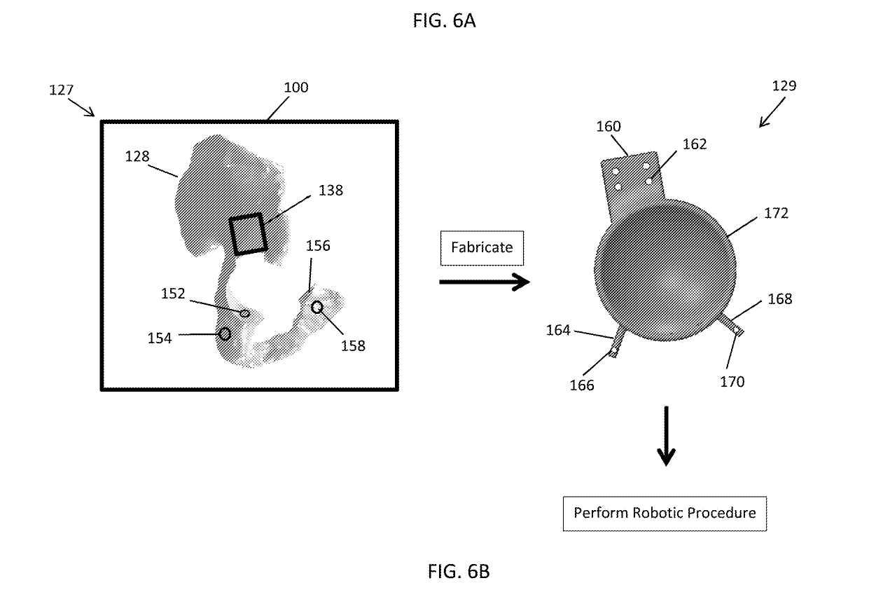

FIGS. 6A and 6B illustrate the progression of designing an implant for acetabular cup reconstruction in accordance with the embodiments described herein. A model of a hemi-pelvis 120 having a degenerated acetabular cup is displayed in the GUI 100 as shown at 120. The user, with the GUI tools, designs an initial implant 132 to replace the degenerated portions of the cup 130. Here, the initial implant design not only includes a geometry to replace the degenerated region, but also includes an acetabular cup portion that interacts with the femur that collectively restore the biomechanics of the subject's joint (e.g. a cup oriented with a 45° inclination and 20° anteversion with respect to the anterior pelvic plane).

A bone density map 134 is displayed in the GUI 100 in the form of brightness values as shown at 122. A key 136 may provide numerical bone property data such as the actual values of the bone density for particular areas on the bone or the stresses experienced at different parts of the bone. The user may locate and highlight a potential stability region 138 to obtain further information regarding the region 138. As shown at 124, the stability region 138 is displayed in greater detail. In particular, the microarchitecture in the stability region 138 is displayed. The outer cortical bone 142 is illustrated as think lines above and below the individual trabeculae. The degree of brightness may indicate the absolute or relative densities of the bone. Each trabecula is characterized by their diameter, length, and structure as well as a degree of brightness representing bone density. The user may select an individual trabecula or a set of trabeculae to obtain numerical property data (e.g. density, diameter, length, bone volume density) which is displayed in a property key 136. The GUI 100 at 124 displays the bone structure and properties pre-simulation and pre-augmentation. A first identified microarchitecture region as shown at 144 illustrates good bone quality with larger diameter trabeculae. This region 144 may have been subject to higher loading conditions, which an FEA using kinematic modelling can reveal. A second microarchitecture region 146 illustrates a poor bone quality region having slimmer trabeculae with higher brightness values corresponding to less density. Likewise, an FEA with kinematic modeling may reveal that this region 146 is subject to less loading. It is possible, however, that the regions having poor bone quality are still subject to higher loads, which might indicate that the bone is osteoporotic. In such a case, the user may augment the implant with features that replace the osteoporotic region, try to shift the loads to regions of higher bone quality, or design features to promote migration to the osteoporotic areas to stimulate bone growth.

The user augments the initial implant with stability, osseointegration features and any retaining components to interact with the located stability region 138. Loading and bone re-modelling simulations are performed to determine how the initial implant and the features affect the loads on the surrounding bone and the re-modelling of the trabecular and cortical bone. For example, a stability feature 148 and osseointegration features 150 are shown at 126 interacting with a portion of the stability region 138. After the simulations, the loads and/or expected bone densities of the cortical and trabeculae are displayed represented. The key 136 may display the bone property data for particular regions of the cortical bone, sets of trabeculae or individual trabeculae. As illustrated at 126, the first microarchitecture region 144′ shows a reduction in bone mass and quality post-simulation. Similarly, the second microarchitecture region 146′ shows and increase in bone mass and quality post-simulation. The simulations and augmented features may be modified iteratively until the desired stability conditions of the final implant are achieved.

FIG. 6B illustrates further steps of the implant design, fabrication and robotic preparation for acetabular cup reconstruction. The final location, size, and geometry of the stability regions are shown at 127, and the fabricated final cup implant is shown at 129. A first stability region 138 for a first augmentation feature 160 is boxed shape that will be partially inset into the bone to a desired depth. The first augmented feature 160 includes a plurality of receiving elements 162 to receive the retaining components. The receiving elements 162 may be for example a straight hole, a threaded hole, a depression, a slot, a hole having an inverse shape of a portion of the retaining component (e.g. a hexagon, a triangle, a square), an interlocking structure having projections in a shape and size resembling an inverse structure of a portion of the retaining component, and combinations thereof. A second stability region 152 for a second augmented feature 164 has a cylindrical shape with a receiving element 166. The location and orientation of the receiving element 166 is designed to receive a retaining component in a particular position and orientation, such that the retaining component and any osseointegration features thereon interacts with the internal structure and density of the bone at that location, represented here as location 154. Likewise, the implant may have a third stability region 156 for a third augmented feature 168 having a cylindrical shape with a receiving element 178. The final implant 129 also includes the cup portion 172 for restoring the biomechanics of the subject's joint.

After the final implant is fabricated, a robotic system can prepare the bone to remove the degenerated tissue and prepare the bone to precisely receive the implant and its features. The robotic system also installs the retaining components in the correct position, orientation and depth to be received in the receiving elements 166 and 170. Since the receiving elements 166 and 170 are not visible once they are inserted into the bone, only a computer-assisted device registered to the bone and implant is capable of locating the precise position of the receiving elements 166 and 170.

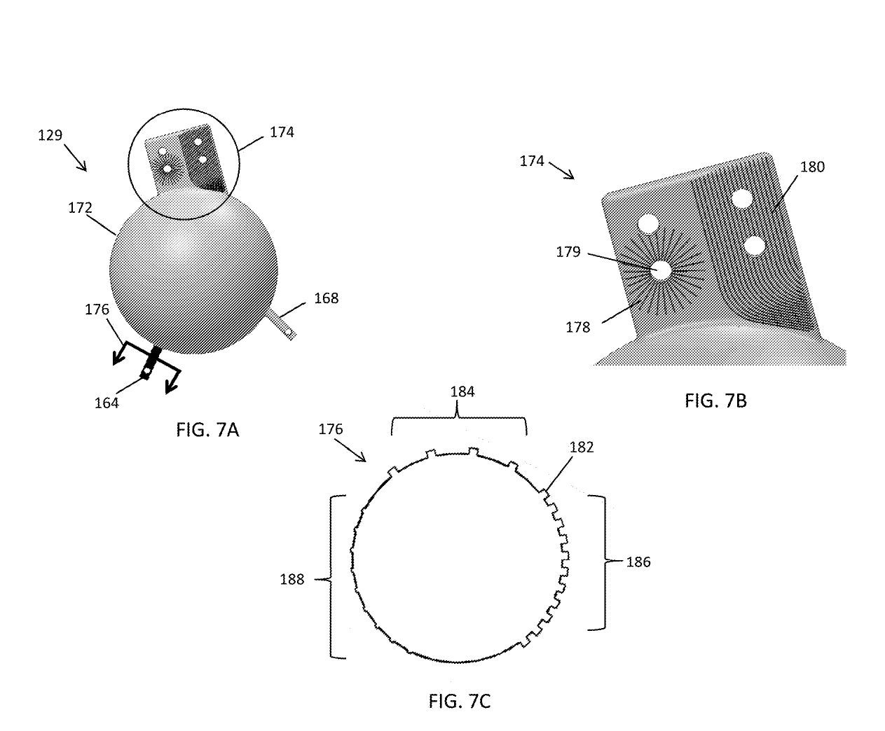

FIGS. 7A-7C illustrate potential osseointegration features designed on the final cup implant 129. FIG. 7A is a rear view of the final cup implant 129 indicating an area of the detailed view 174 shown in FIG. 7B, and a cross-section 176 of the view shown in FIG. 7C. The detailed view 174 illustrates two examples of osseointegration features 178 and 180. A first osseointegration feature 178 includes a set of channels in a radial pattern emanating from a receiving element 179. The first osseointegration feature 178 is designed to promote the migration and subsequent formation of bone towards the intersection of a retaining component and receiving element 179. A second osseointegration feature 180 includes a set of channels oriented perpendicular to the direction of the loads modelled during the simulations. The width and depth of the channels promote osteoblast adhesion and bone formation to aid in the re-structuring of the trabeculae due to the newly experienced loads in vivo.

The cross-section view 176 illustrates examples of osseointegration features on the cylindrical stability feature 164. The osseointegration features include sets of projections 182 varying in distribution, spacing, height and width, which correspond to the microarchitecture and simulations performed during implant design. A first set of projections 184 include moderately spaced projections to promote cell migration, which may be beneficial for promoting bone growth from a high-bone quality region pre-implantation and less load post-implantation to a low-bone quality region pre-implantation and high load post-implantation. A second set of projections 186 have projections closely spaced together to promote osteoblast differentiation and bone formation. A third set of projections 188 may have less height to interact with the hard cortical bone, without invasively damaging the cortical bone. The projections may directly intersect with the bone when implanted creating an interference fit. For example, the robotic system may drill a cylindrical hole in the bone, where the hole has a diameter just less than the diameter of the cylindrical stability feature 164. Or, the bone may be prepared to interdigitate with the projections using the methods as described herein, such as a final surface cut with a tool having a negatively matched pattern.

### Example 3: Tibial Implant Design for ACL Retaining TKA

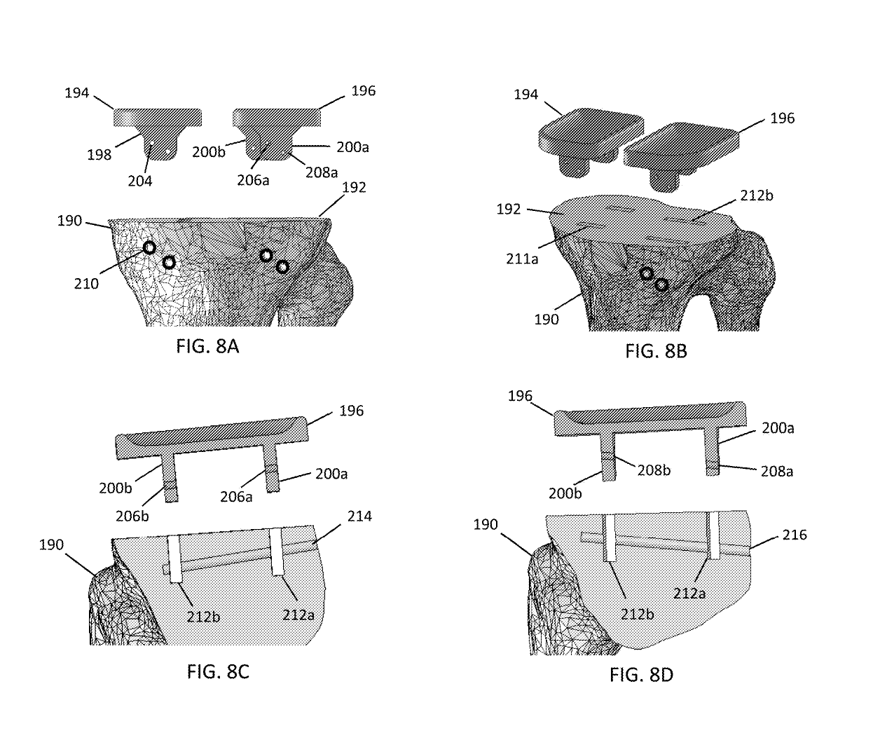

FIGS. 8A-8D illustrate a tibial implant design for an anterior cruciate ligament (ACL) retaining total knee arthroplasty (TKA) in accordance with the embodiments described herein. Currently, ACL retaining tibial implants are separated into a medial implant compartment and a lateral implant compartment that are partially connected at their anterior end by a support. Due to this bi-compartmental structure, each implant has less bone to grasp to fix into the bone. This makes the implants susceptible to failure primarily due to shearing forces. To provide a bi-compartment ACL retaining tibial implant that improves implant longevity, the implant needs stability and osseointegration features tailored to each specific subject. One potential design is shown in FIGS. 8A-8D.

The design of the tibial implant begins by obtaining a model of the femur (not shown) and the tibia 190. At least one of the size, type, geometry and position of the femoral implant (not shown) and a medial tibial compartment 194 and a lateral tibial compartment 196 is determined to restore the biomechanics of the subject's anatomy (e.g. restore the mechanical axis of the subject's leg). The size, type, geometry and position of the tibial compartments define the volume, position and orientation of the tibial bone cuts 192 to receive the tibial compartment. In particular, the fabrication instructions used to fabricate the medial tibial compartment 194 and lateral tibial compartment 196 are used to generate a cut-file for the robotic surgical system to precisely prepare the tibia 190 to receive the compartments. Although FIGS. 8A-8D depict the tibial bone cut 192 as a planar surface across the entire tibia, in reality, the middle portion of the tibia is preserved to retain the ACL and the bone cuts are prepared to negatively match the outer geometry of the tibial compartments 194 and 196.

The user locates stability regions in the tibia model 190 using any of the aforementioned methods and the medial compartment 194 and lateral compartment 196 are augmented to interact with the located stability regions. Here, the medial compartment 194 includes a medial anterior keel 198 and a medial posterior keel positioned in-line with the medial anterior keel 198 from an anterior view perspective as shown in FIG. 8A. The lateral compartment 196 includes a lateral anterior keel 200a and a lateral posterior keel 200b, with the posterior keel 200b medially offset from the lateral anterior keel 200a. To promote osseointegration the keel 198 has an anodized surface coated with slow release VEGF. Two representative bone cuts to receive the augmented features are shown in FIG. 8B, of which one cut 211a receives the medial anterior keel 198 and a second bone cut 212b receives the lateral posterior keel 200b. The other bone cuts are created accordingly to receive the other augmented features. In some embodiments, the cuts include autologous bone chips to promote osseointegration.

The keels are designed with receiving elements to receive retaining components there through. By inspecting the bone quality and performing the simulations, the retaining components are designed to intersect and interact with the bone in-between the outer surface of the bone and the receiving elements. The position and orientation of the retaining components are also designed to improve the stability of the implant to account for the shear forces and other loading conditions experienced on the tibial components in vivo. For example, the medial tibial keels include a receiving element 204 that receives a retaining component via a drill hole 210. The other drill holes as outlined by the black circles as shown in FIGS. 8A and 8B indicate the position for the other retaining components.

The retaining components may be designed similar to that as shown in FIGS. 3A and 3B, but adapted to the subject's specific anatomy. In particular, the position and orientation of the receiving elements and the position and orientation for inserting the retaining component to intercept with the receiving elements are highly dependent on the subject's bone quality and the located stability regions. FIG. 8C illustrates the tibia 190 and the lateral tibial component 196 cross-sectioned along an axis that connects anteromedial receiving element 206a and posteromedial receiving element 206b. An anterior bone cut 212a receives the lateral anterior keel 200a, and a posterior bone cut 212b receives the lateral posterior keel 200b. The receiving elements 206a and 206b are oriented such that when a retaining component is inserted in a medial drill hole 214, or directly inserted into the bone along that axis, the retaining component and any osseointegration features thereon interact with the bone and microarchitecture therebetween. The path of the retaining component when inserted may also push or displace one bone quality type in one region to another region to promote bone formation and ultimately the stability of the implant with the native bone. FIG. 8D illustrates the tibia 190 and the lateral tibial compartment 196 cross-sectioned along an axis that connects the anterolateral receiving element 208a and the posterolateral receiving element 208b. Similarly, the receiving elements 208 and the corresponding retaining component inserted into the bone is positioned and oriented to interact with the bone along the path 216. Due to these offset and subject specific insertion paths for the retaining components, only a computer-assisted device having the bone and the pre-operative plan registered to the computer-assisted device can successfully align and insert these components to exploit the advantages of these features.

### Example 4: Implant Design and Robotics for Spinal Surgery

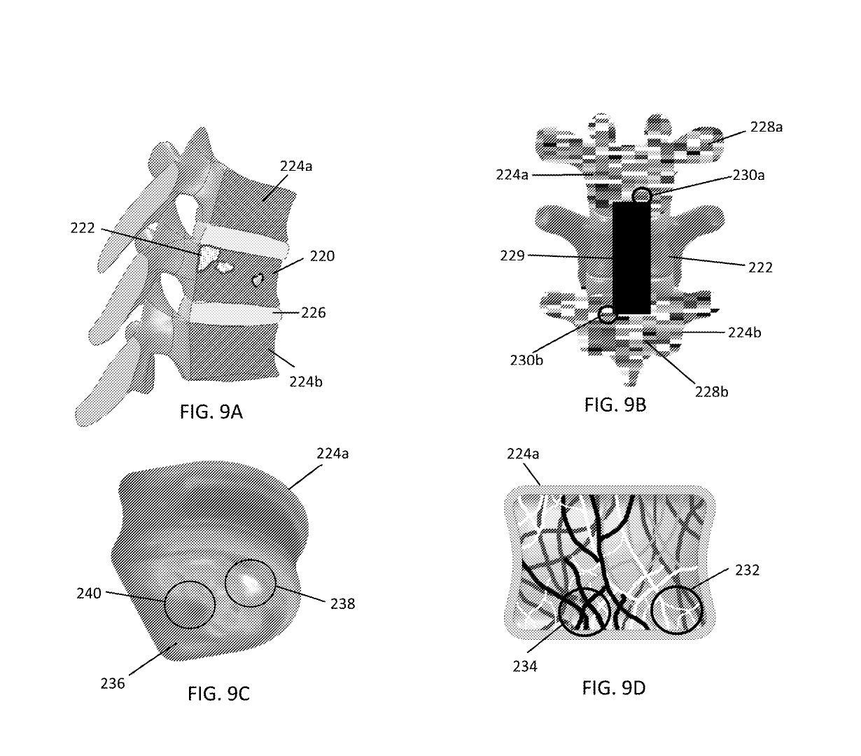

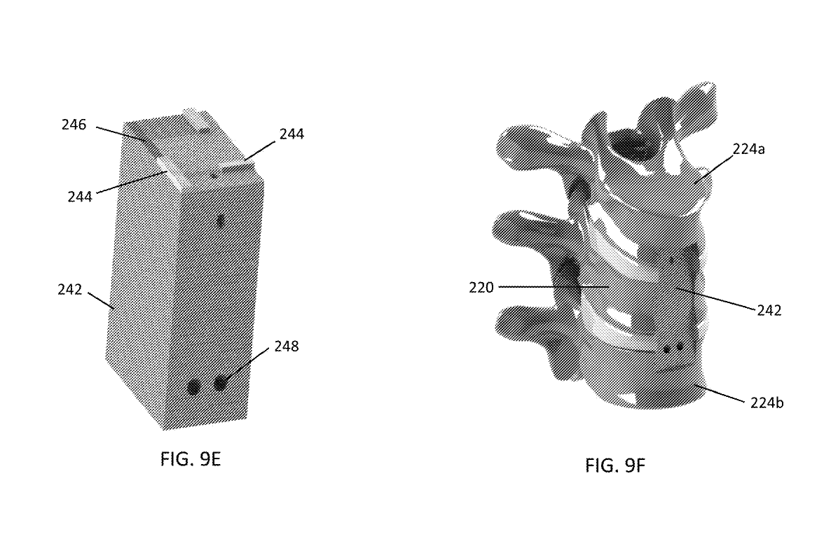

Embodiments of the present invention may be directly applied to spinal surgeries including complete and partial corpectomy (i.e. vertebral body replacement), discectomies, intervertebral fusions, and vertebroplasties. FIGS. 9A-9F illustrate an example of designing an implant for replacing a portion of a vertebral body having cancerous tumors. FIG. 9A is a cross-section of a portion of a vertebrae model depicting a targeted vertebral body 220 having cancerous tumors 222 compressing the spinal cord. An upper vertebra 224a and a lower vertebra 224b are shown above and below the targeted vertebral body 220, respectively, with intervertebral discs 226 therebetween. The goal of the surgery is to decompress the spinal cord, excise the tumorous growths, and stabilize the spinal column by fusing the upper and lower vertebra 224 with the implant. A current type of vertebral body implant generally includes a bone graft placed within an adjustable cage to promote arthrodesis. The caged implants are subjectively placed between the vertebras 224 and adjusted to stabilize the spine. However, this subjective placement does not account for the varying bone quality of the vertebra 224 or the stresses and loads caused by the vertebra 224, which can have a significant effect on how the bones fuse to the implant and the time required to fully stabilize that portion of the spine.

To overcome the current limitations, the user first designs an initial implant 229 that can replace the region that will be removed to decompress the spinal cord and excise the tumors. Subsequently, the user identifies stability regions 230 near the initial implant interface using a bone density map 228 as shown in FIG. 9B. FIG. 9C illustrates a loading/stress map 236 on the bottom endplate of the upper vertebra 224a from an FEA performed on the subject's spine with the initial implant 229 in place. The loading/stress map 236 indicates a region of higher loading 238 and a region of lower loading 240. A similar load/stress map is also provided, although not shown, for the upper endplate of the lower vertebra 224b. The higher loading regions indicate where bone re-modelling and bone formation may occur first. The lower loading regions may require the recruitment or migration of cells to initially promote osseointegration. As such, the stability and osseointegration features can be augmented to the initial implant 229 accordingly.

FIG. 9D illustrates the microarchitecture of the upper vertebra 224a showing a region of dense and larger diameter trabeculae 234 and a region of less dense a smaller diameter trabeculae 232. The user can augment the initial implant with stability and osseointegration features to promote bone growth in a particular direction or in a specific quantity to promote the fusion of the vertebra to the implant and to restore the natural loads experienced at this portion of the spine. The FEA and bone remodeling simulations can be performed at any stage during this implant design procedure to optimize the placement, geometry and size of the augmented stability and osseointegration features.

FIG. 9E illustrates an example of a final implant 242. Preferably, the final implant 242 is made of PEKK because of its biocompatibility and superior compressive mechanical properties to withstand the pressures of the spine. The final implant design 242 includes stability features 244 and osseointegration features 246 that interact with congruent features robotically prepared on the adjacent vertebra 224. The final implant 242 may also include receiving elements 248 to receive retaining components. The POSE of the receiving elements 248 and the design of the retaining components are intended to anchor into the adjacent vertebra 224 and promote osseointegration or cell migration to the implant-bone interface. FIG. 9F illustrates the final position of the implant 242 in the spine.

In a particular embodiment, the final implant 242 is made from an autologous bone graft harvested intra-operatively. A computer-assisted surgical system may harvest the bone graft from the donor site to precisely match the geometry of the final implant 242. Or, the system may harvest a slightly oversized graft and subsequently create the stability and osseointegration features thereon. In another embodiment, the final implant 242 is made of an allogenic bone graft. The geometry of the allogenic bone graft can be prepared pre-operatively according to the final implant design.

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A method of creating an augmented implant for a subject's bone or joint, the subject's bone or joint having a required region for removal, the method comprising:
obtaining a virtual bone model of the subject's bone or joint, the bone model having bone property data for the subject's bone or joint including at least topology, density, and microarchitecture data;
determining at least one of a size, a type, a geometry and a position for a virtual implant model with respect to the bone or joint model to replace the region for removal using a processor;
locating a stability region in the bone or joint surrounding the position of the implant model based on the bone property data;
augmenting the implant model with one or more stability features to interact with the stability region to improve implant stability using said processor or another processor; and
creating the augmented implant.

2. The method of claim 1, wherein locating the stability region further comprises:
selecting a region on the bone model having a higher bone density relative to other regions on the bone model;
displaying the microarchitecture of the bone in the region;
simulating forces experienced on the region based on the position of the implant model;
displaying loading conditions existing on the microarchitecture from the simulation; and
locating a second stability region based on the loading conditions.

3. The method of claim 1 further comprising simulating a re-modelling of the bone microarchitecture after augmentation and modifying the stability feature based on at least one of an increase in trabecular bone density or a decrease in trabecular bone density.

4. The method of claim 1 further comprising constraining the position of the implant model with respect to the bone model prior to the augmentation.

5. The method of claim 1 further comprising:
locating an internal stability region between a stability feature and an outer surface of the bone model;
defining an axis through the outer surface of the bone model, the inner stability region, and the stability feature;
creating a receiving element on the stability feature through the axis; and
designing a retaining component to interfere with at least a portion of the stability region along the axis and wherein a portion of the retaining component is received in the receiving element.

6. The method of claim 5 wherein the retaining component is a bone screw, a bone pin, or a bone nail and the receiving element is configured to receive a portion of the retaining component.

7. The method of claim 5 further comprising augmenting the retaining component with an osseointegration feature to interact with the microarchitecture of the bone along the length of at least a portion of the retaining component.

8. The method of claim 1 further comprising augmenting at least one of the implant model and the one or more stability features with an osseointegration feature to promote at least one of bone formation, osteoblast differentiation, or osteoblast migration.

9. The method of claim 1 wherein the osseointegration feature is in the form of a channel, a ridge, a groove, or a projection.

10. The method of claim 1 wherein augmenting the implant model with stability features further comprises creating a series of interdigitating stability features so as to respect the three-dimensional (3-D) anatomical relationship with the bone model.

11. The method of claim 1 further comprising, generating fabrication instructions for the implant based on the geometry of the implant and the one or more augmented features, and sending the fabrication instructions to a manufacturer to fabricate the implant for use in surgery.

12. The method of claim 11 further comprising, generating at least a portion of a cut-file for a computer-assisted surgical device based on a set of points extracted from the fabrication instructions.

13. The method of claim 12 further comprising, registering the subject's bone to the surgical system, and preparing the bone according to the cut-file.

14. The method of claim 13 further comprising, registering the retaining component to the surgical system, and implanting, with the surgical system, the retaining component to a desired depth and a rotational position as determined during the augmentation of the osseointegration feature.

15. The method of claim 1 further comprising coating the augmented implant with an osseointegration promoting growth factor.

16. The method of claim 15 where said osseointegration promoting growth factor is provided in a slow release matrix.

17. The method of claim 1 further comprising promoting osseointegration of the augmented implant through a surface modification including at least one coating with hydroxyapatite, roughening on a scale that promotes osteoblast infiltration, and growing thicker than native oxides.
